(19) United States
(12) Patent Application Publication
(10) Pub. No.: US 2010/0158919 A1
(43) Pub. Date: Jun. 24, 2010

(54) Pharmaceutical Composition

(76) Inventors: Isabelle Bauer Dauphin, Kembs (FR); Hanns-Christian Mahler, Basel (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 12/636,964

(22) Filed: Dec. 14, 2009

(30) Foreign Application Priority Data
Dec. 19, 2008 (EP) 08172314.0

(51) Int. Cl. A61K 39/395; A61P 43/00

(52) U.S. Cl. 424/142.1

(57) ABSTRACT

The present invention relates to an anti-IGF-1R human monoclonal antibody formulation, a process for the preparation of said formulation and uses thereof

### PRIORITY TO RELATED APPLICATION(S)

This application claims the benefit of European Patent Application No. 08172314.0, filed Dec. 19, 2008, which is hereby incorporated by reference in its entirety.

The present invention relates to an anti-IGF-1R human monoclonal antibody formulation, a process for the preparation of said formulation and uses thereof.

In one aspect, the invention relates to an IGF-1R antibody formulation comprising:

about 20 to about 60 mg/mL huMAb-IGF-1R,

about 5 to about 50 mM of a buffer,

at a pH in the range from about 4.5 to about 7.0.

The IGF-IR (type 1 insulin-like growth factor receptor), has been implicated in promoting oncogenic transformation, growth, and survival of cancer cells. High levels of expression of IGF-IR have been reported in a broad range of human malignancies. In addition, high levels of IGF-I and IGF-II expression have been noted in tumors and associated stromal cells and may stimulate cancer cell growth in an autocrine or paracrine manner. Epidemiological studies have correlated upper quintile plasma levels of IGF-I with increased risk for prostate, colon, lung, and breast cancer. In addition to its role in proliferation of cancer cells, IGF-IR protects cells from apoptosis caused by growth factor deprivation, anchorage independence, or cytotoxic drug treatment.

One promising strategy to inhibit the function of IGF-IR in cancer cells is to apply human anti-IGF-IR antibodies that bind to the extracellular domains of IGF-IR and inhibit receptor activation. Such an antagonistic, fully human, monoclonal antibody, designated huMAb-IGF-1R, has been developed which binds specifically to the human insulin-like growth factor I receptor (IGF-IR) and inhibits signal transduction and the proliferation functions of the receptor in cancer cells.

The antibody comprised in the formulation of the invention has been first described in PCT patent application No. WO2005/005635 of which the Applicant is proprietor and the content of which, especially the claims is incorporated herein by reference. As described in WO2005/005635, said antibody is binding to IGF-IR and inhibiting the binding of IGF-I and IGF-II to IGF-IR, and is characterized in that it:
a) is of IgG1 isotype,
b) shows a ratio of IC₅₀ values of inhibition of the binding of IGF-I to IGF-IR to the inhibition of binding of IGF-II to IGF-IR of 1:3 to 3:1,
c) inhibits for at least 80%, preferably at least 90%, at a concentration of 5 nM IGF-IR phosphorylation in a cellular phosphorylation assay using HT29 cells in a medium containing 0.5% heat inactivated fetal calf serum (FCS) when compared to such an assay without said antibody, and
d) shows no IGF-IR stimulating activity measured as pkB phosphorylation at a concentration of 10 μM in a cellular phosphorylation assay using 3T3 cells providing 400,000 to 600,000 molecules IGF-IR per cell in a medium containing 0.5% heat inactivated fetal calf serum (FCS) when compared to such an assay without said antibody.

### BRIEF DESCRIPTION OF THE DRAWINGS

FIG. 1 shows the effect of changing pH and temperature on HMW soluble aggregates in formulations of the invention.

FIG. 2 shows the effect of changing pH and temperature on LMW fragments in formulations of the invention.

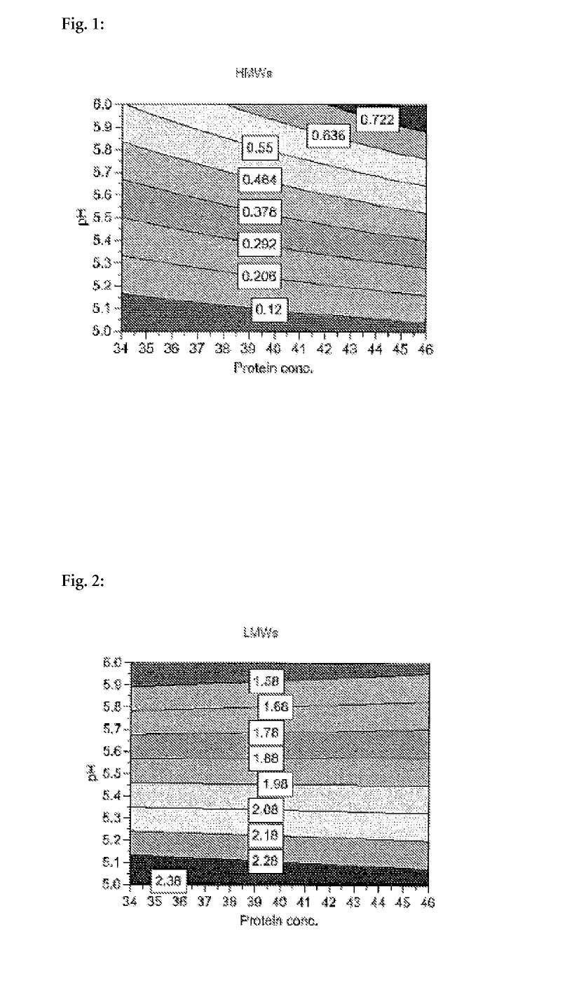

### DETAILED DESCRIPTION OF THE INVENTION

Antibodies comprised in the formulation according to the invention show benefits for patients in need of antitumor therapy and provide reduction of tumor growth and a significant prolongation of the time to progression. The antibodies comprised in the formulation according to the invention have new and inventive properties causing a benefit for a patient suffering from a disease associated with an IGF deregulation, especially a tumor disease. The antibodies comprised in the formulation of the invention are characterized by the abovementioned properties. The properties are therefore especially specific binding to IGF-IR, inhibiting the binding of IGF-I and IGF-II to IGF-IR at the abovementioned ratio, being of IgG1 isotype, and not activating the IGF-IR signaling even in IGF-IR overexpressing cells at a 200-fold concentration of its IC₅₀ value. Antibodies having no “IGF-I mimetic activity” provide a strong advantage when used as a therapeutic agent.

The term “about” denotes a minor±variability. In general, it is meant to denote a variability of 0.5% to 2%.

The term “anti-IGF-1R human monoclonal antibody” or “huMAb IGF-IR” denotes an antibody as described and claimed in WO2005/005635, the content of which, especially the claims, is incorporated herein by reference.

The term “antibody” encompasses the various forms of antibodies including but not being limited to whole antibodies, human antibodies, humanized antibodies and genetically engineered antibodies like monoclonal antibodies, chimeric antibodies or recombinant antibodies as well as fragments of such antibodies as long as the characteristic properties according to the invention are retained.

“Antibody fragments” comprise a portion of a full length antibody, generally at least the antigen binding portion or the variable region thereof. Examples of antibody fragments include diabodies, single-chain antibody molecules, immunotoxins, and multispecific antibodies formed from antibody fragments. In addition, antibody fragments comprise single chain polypeptides having the characteristics of a VH chain, namely being able to assemble together with a VL chain or of a VL chain binding to IGF-1R, namely being able to assemble together with a VH chain to a functional antigen binding pocket and thereby providing the property of inhibiting the binding of IGF-I and IGF-II to IGF-IR.

“Antibody fragments” also comprises such fragments which per se are not able to provide effector functions (ADCC/CDC) but provide this function in a manner according to the invention after being combined with appropriate antibody constant domain(s).

The terms “monoclonal antibody” or “monoclonal antibody composition” as used herein refer to a preparation of antibody molecules of a single amino acid composition. Accordingly, the term “human monoclonal antibody” refers to antibodies displaying a single binding specificity which have variable and constant regions derived from human germline immunoglobulin sequences. In one embodiment, the human monoclonal antibodies are produced by a hybridoma which includes a B cell obtained from a transgenic non-human animal, e.g. a transgenic mouse, having a genome comprising a human heavy chain transgene and a light human chain transgene fused to an immortalized cell.

The term “chimeric antibody” refers to a monoclonal antibody comprising a variable region, i.e., binding region, from one source or species and at least a portion of a constant region derived from a different source or species, usually prepared by recombinant DNA techniques. Chimeric antibodies comprising a murine variable region and a human constant region are especially preferred. Such murine/human chimeric antibodies are the product of expressed immunoglobulin genes comprising DNA segments encoding murine immunoglobulin variable regions and DNA segments encoding human immunoglobulin constant regions. Other forms of “chimeric antibodies” encompassed by the present invention are those in which the class or subclass has been modified or changed from that of the original antibody. Such “chimeric” antibodies are also referred to as “class-switched antibodies.” Methods for producing chimeric antibodies involve conventional recombinant DNA and gene transfection techniques now well known in the art. See, e.g., Morrison, S. L., et al., Proc. Natl. Acad. Sci. USA 81 (1984) 6851-6855; U.S. Pat. Nos. 5,202,238 and 5,204,244.

The term “humanized antibody” refers to antibodies in which the framework or “complementarity determining regions” (CDR) have been modified to comprise the CDR of an immunoglobulin of different specificity as compared to that of the parent immunoglobulin. In a preferred embodiment, a murine CDR is grafted into the framework region of a human antibody to prepare the “humanized antibody.” See, e.g., Riechmann, L., et al., Nature 332 (1988) 323-327; and Neuberger, M. S., et al., Nature 314 (1985) 268-270. Particularly preferred CDRs correspond to those representing sequences recognizing the antigens noted above for chimeric and bifunctional antibodies.

The term “human antibody”, as used herein, is intended to include antibodies having variable and constant regions derived from human germline immunoglobulin sequences. The variable heavy chain is preferably derived from germline sequence DP-50 (GenBank L06618) and the variable light chain is preferably derived from germline sequence L6 (GenBank X01668). The constant regions of the antibody are constant regions of human IgG1 type. Such regions can be allotypic and are described by, e.g., Johnson, G., and Wu, T. T., Nucleic Acids Res. 28 (2000) 214-218 and the databases referenced therein and are useful as long as the properties of induction of ADCC and preferably CDC according to the invention are retained.

The term “recombinant human antibody”, as used herein, is intended to include all human antibodies that are prepared, expressed, created or isolated by recombinant means, such as antibodies isolated from a host cell such as an SP2-0, NS0 or CHO cell or from an animal (e.g. a mouse) that is transgenic for human immunoglobulin genes or antibodies expressed using a recombinant expression vector transfected into a host cell. Such recombinant human antibodies have variable and constant regions derived from human germline immunoglobulin sequences in a rearranged form. The recombinant human antibodies according to the invention have been subjected to in vivo somatic hypermutation. Thus, the amino acid sequences of the VH and VL regions of the recombinant antibodies are sequences that, while derived from and related to human germline VH and VL sequences, may not naturally exist within the human antibody germline repertoire in vivo.

The term “binding to IGF-IR” as used herein means the binding of the antibody to IGF-IR in an in vitro assay, preferably in a binding assay in which the antibody is bound to a surface and binding of IGF-IR is measured by Surface Plasmon Resonance (SPR). Binding means a binding affinity (K_{D}) of 10⁻⁸ M or less, preferably 10⁻¹³ to 10⁻⁹ M. The binding affinity is determined with a standard binding assay, such as surface plasmon resonance technique (Biacore®).

The term “nucleic acid molecule”, as used herein, is intended to include DNA molecules and RNA molecules. A nucleic acid molecule may be single-stranded or double-stranded, but preferably is double-stranded DNA.

The “constant domains” are not involved directly in binding the antibody to an antigen but are involved in the effector functions (ADCC, complement binding, and CDC). The constant domain of an antibody according to the invention is of the IgG1 type. Human constant domains having these characteristics are described in detail by Kabat et al., Sequences of Proteins of Immunological Interest, 5th Ed. Public Health Service, National Institutes of Health, Bethesda, Md. (1991), and by Brüggemann, M., et al., J. Exp. Med. 166 (1987) 1351-1361; Love, T. W., et al., Methods Enzymol. 178 (1989) 515-527. Examples are shown in SEQ ID NOS:5 to 8 in WO2005/005635. Other useful and preferred constant domains are the constant domains of the antibodies obtainable from the hybridoma cell lines deposited with DSMZ for this invention. The constant domains useful in the invention provide complement binding. ADCC and optionally CDC are provided by the combination of variable and constant domains.

The “variable region” (variable region of a light chain (VL), variable region of a heavy chain (VH)) as used herein denotes each of the pair of light and heavy chains which is involved directly in binding the antibody to the antigen. The domains of variable human light and heavy chains have the same general structure and each domain comprises four framework (FR) regions whose sequences are widely conserved, connected by three “hypervariable regions” (or complementarity determining regions, CDRs). The framework regions adopt a β-sheet conformation and the CDRs may form loops connecting the β-sheet structure. The CDRs in each chain are held in their three-dimensional structure by the framework regions and form together with the CDRs from the other chain the antigen binding site. The antibody heavy and light chain CDR3 regions play a particularly important role in the binding specificity/affinity of the antibodies according to the invention and therefore provide a further object of the invention.

The terms “hypervariable region” or “antigen-binding portion of an antibody” when used herein refer to the amino acid residues of an antibody which are responsible for antigen-binding. The hypervariable region comprises amino acid residues from the “complementarity determining regions” or “CDRs”. “Framework” or “FR” regions are those variable domain regions other than the hypervariable region residues as herein defined. Therefore, the light and heavy chains of an antibody comprise from N— to C-terminus the domains FR1, CDR1, FR2, CDR2, FR3, CDR3, and FR4. Especially, CDR3 of the heavy chain is the region which contributes most to antigen binding. CDR and FR regions are determined according to the standard definition of Kabat et al., Sequences of Proteins of Immunological Interest, 5th Ed. Public Health Service, National Institutes of Health, Bethesda, Md. (1991)) and/or those residues from a “hypervariable loop”.

Binding to IGF-IR can be investigated by a BIAcore assay (Pharmacia Biosensor AB, Uppsala, Sweden). The affinity of the binding is defined by the terms ka (rate constant for the association of the antibody from the antibody/antigen complex), kd (dissociation constant), and K_{D} (kd/ka). The antibodies according to the invention show a K_{D} of 10⁻¹⁰ M or less.

The binding of IGF-I and IGF-II to IGF-IR is also inhibited by the antibodies according to the invention. The inhibition is measured as IC₅₀ in an assay for binding of IGF-I/IGF-II to IGF-IR on tumor cells. In such an assay, the amount of radiolabeled IGF-I or IGF-II or IGF-IR binding fragments thereof bound to the IGF-IR provided at the surface of said tumor cells (e.g. HT29) is measured without and with increasing concentrations of the antibody. The IC₅₀ values of the antibodies according to the invention for the binding of IGF-I and IGF-II to IGF-IR are no more than 2 nM and the ratio of the IC₅₀ values for binding of IGF-I/IGF-II to IGF-IR is about 1:3 to 3:1. IC₅₀ values are measured as average or median values of at least three independent measurements. Single IC₅₀ values may be out of the scope.

The term “inhibiting the binding of IGF-I and IGF-II to IGF-IR” as used herein refers, e.g., to inhibiting the binding of I¹²⁵-labeled IGF-I or IGF-II to IGF-IR presented on the surface of HT29 (ATCC HTB-38) tumor cells in an in vitro assay. Inhibiting means an IC₅₀ value of 2 nM or lower.

Therapeutic formulations of the antibodies used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed.

The term “surfactant” as used herein denotes a pharmaceutically acceptable surface-active agent. In the formulation of the invention, the amount of surfactant is described as a percentage expressed in weight/volume. The most commonly used weight/volume unit is mg/mL. Suitable pharmaceutically acceptable surfactants comprise but are not limited to polyethylen-sorbitan-fatty acid esters, polyethylene-polypropylene glycols, polyoxyethylene-stearates and sodium dodecyl sulphates. Preferred polyethylen-sorbitan-are polyethylen(20)-sorbitan-esters (synonym to polysorbate 20, sold under the trademark Tween 20™) and polyoxyethylene(20)sorbitanmonooleate (synonym to polysorbate 80 sold under the trademark Tween 80™). Preferred polyethylene-polypropylene glycols are those sold under the names Pluronic® F68 or Poloxamer 188™. Preferred polyoxyethylene-stearates are those sold under the trademark Myrj™. Preferred polyoxyethylene monolauryl ether are those sold under the trademark Brij™. When polyethylen-sorbitan-polyethylen(20)-sorbitan-esters (Tween 20™) and polyoxyethylene(20)sorbitanmonooleate (Tween 80™) are used they are preferably used in an amount of about 0.01% to about 0.06%, more preferably of about 0.02% to about 0.04% and most preferably about 0.03% w/v.

When amounts are expressed as “mM” herein, it means the amount of a given agent that will yield the recited concentration of agent in mM.

The term “buffer” as used herein denotes a pharmaceutically acceptable buffer. Suitable pharmaceutically acceptable buffer comprise but are not limited to histidine-buffers, citrate-buffers, succinate-buffers, acetate-buffers and phosphate-buffers. Preferred buffers comprise L-histidine or mixtures of L-histidine with L-histidine hydrochloride with isotonicity agents and potentially pH adjustment with an acid or a base known in the art. The abovementioned histidine-buffers are generally used in an amount of about 1 mM to about 100 mM, preferably of about 5 mM to about 50 mM and still more preferably of about 20 mM. Independently from the buffer used, the pH will be adjusted at a value comprising about 4.5 to about 7.0 and preferably about 5.0 to about 6.0 and most preferably about 5.5 by adjustment with an acid or base known in the art or by using adequate mixtures of buffer components or both.

The term “isotonicity agents” as used herein denotes pharmaceutically acceptable isotonicity agents. Isotonicity agents are used to provide an isotonic formulation. An isotonic formulation is liquid or liquid reconstituted from a solid form, e.g. a lyophilized form and denotes a solution having the same tonicity as some other solution with which it is compared, such as physiologic salt solution and the blood serum. Suitable isotonicity agents comprise but are not limited to salts, including but not limited to sodium chloride(NaCl) or potassium chloride, sugars including but not limited to glucose, sucrose, trehalose or and any component from the group of amino acids, sugars, salts and combinations thereof. Isotonicity agents are generally used in a total amount of about 5 mM to about 350 mM.

The term “liquid” as used herein in connection with the formulation according to the invention denotes a formulation which is liquid at a temperature of at least about 2 to about 8° C. Unless otherwise noted, the term “formulation” herein refers to a liquid formulation.

The term “lyophilized” as used herein in connection with the formulation according to the invention denotes a formulation which is dried by freezing the formulation and subsequently subliming the ice from the frozen content by any freeze-drying methods known in the art, for example commercially available freeze-drying devices. When amounts of lyophilized formulation components are set forth, they refer to the concentration of the component after the lyophilized formulation has been reconstituted in water.

The term “salts” as used herein denotes a salt in an amount of about 1 mM to about 500 mM. Non-limiting examples of salts include salts of any combinations of the cations sodium potassium, calcium or magnesium with anions chloride, phosphate, citrate, succinate, sulphate or mixtures thereof.

The term “amino acid” as used herein denotes an amino acid in an amount of about 1 to about 200 mg/mL comprising but not limited to arginine, glycine, ornithine, lysine, histidine, glutamic acid, asparagic acid, isoleucine, leucine, alanine, phenylalanine, tyrosine, tryptophane, methionine, serine, proline. Preferred is methionine. More preferred is methionine at about 5 mM to about 15 mM. Most preferred is methionine at about 10 mM. Also preferred is arginine HCl, more preferred is arginine HCl at about 100 mM to about 200 mM. Most preferred is arginine HCl at about 150 mM.

The term “sugar” as used herein denotes a pharmaceutically acceptable sugar used in an amount of about 25 mM to about 500 mM. Suitable sugars comprise but are not limited monosaccharides and disaccharides. Non-limiting examples of sugars according to the invention include trehalose, sucrose, mannitol, sorbitol, lactose, glucose, mannose, maltose, galactose, fructose, sorbose, raffinose, glucosamine, N-methylglucosamine (also referred to as “meglumine”), galactosamine and neuraminic acid and combinations thereof. Preferred is trehalose. More preferred is trehalose at about 200 mM to about 300 mM. Most preferred is trehalose at about 240 mM.

The term “stabilizer” refers to pharmaceutically acceptable stabilizers, like for example but not limited to amino acids and sugars as described in the above sections as well as commercially available cyclodextrins and dextrans of any kind and molecular weight as known in the art. Preferred are amino acids and/or sugars.

The term “antioxidant” denotes a pharmaceutically acceptable antioxidant. This may include excipients such as methionine, benzylalcohol or any other excipient used to minimize oxidation.

As mentioned above, in one aspect, the invention relates to an IGF-1R antibody formulation comprising:

about 20 to about 60 mg/mL huMAb-IGF-1R,

about 5 to about 50 mM of a buffer,

at a pH in the range from about 4.5 to about 7.0.

A preferred formulation according to the invention comprises:

about 30 to about 50 mg/mL huMAb-IGF-1R,

about 10 to about 30 mM of a buffer,

at a pH in the range from about 5.0 to about 6.5.

A preferred formulation according to the invention further comprises about 0.01% to about 0.06% of at least one surfactant.

A preferred formulation according to the invention further comprises at least one stabilizer.

In a preferred formulation according to the invention, the stabilizers are selected from the group of sugars and amino acids.

The formulation of the invention can comprise a sugar in an amount of about 25 mM to about 500 mM. Suitable sugars can be selected from the group consisting of trehalose, saccharose, lactose, glucose, mannose, maltose, galactose, fructose, sorbose, raffinose, glucosamine, N-Methylglucosamine, galactosamine, neuraminic acid and combinations thereof. In a more preferred formulation according to the invention, the stabilizer is trehalose. In an even more preferred formulation according to the invention, the formulation comprises about 200 mM to about 300 mM trehalose.

In another more preferred formulation according to the invention, the stabilizer is an amino acid. Preferred is methionine. In another more preferred formulation according to the invention, the formulation comprises about 10 mM to about 30 mM methionine.

In another more preferred formulation according to the invention, the formulation comprises trehalose and methionine. In another more preferred formulation according to the invention, the formulation comprises about 230 mM to 250 mM trehalose and about 5 mM to 15 mM methionine.

Another more preferred formulation according to the invention comprises:

about 35 to about 45 mg/mL huMAb-IGF-1R,

at least one further stabilizer, and

about 0.02 to about 0.04% of at least one surfactant, and

about 15 to about 25 mM of a buffer,

at a pH in the range from about 5.0 to about 6.0.

Another more preferred formulation according to the invention, wherein the formulation comprises about 230 mM to 250 mM trehalose and about 5 mM to 15 mM methionine.

Another more preferred formulation according to the invention comprises:

about 40 mg/mL huMAb-IGF-1R,

about 240 mM trehalose, and

about 0.03 of polysorbate 20 or polysorbate 80, and

about 20 mM of L-histidine,

at a pH of about 5.5.

In a most preferred formulation according to the invention, the formulation as disclosed in the paragraph above further comprises about 10 mM methionine.

Another more preferred formulation according to the invention comprises:

about 40 mg/mL huMAb-IGF-1R,

about 240 mM Sucrose, and

about 0.03 of polysorbate 20 or polysorbate 80, and

about 20 mM of L-histidine,

at a pH of about 5.5.

In a most preferred formulation according to the invention, the formulation as disclosed in the paragraph above further comprises about 10 mM methionine.

A preferred formulation according to the invention, is in a liquid form, in a lyophilized form or in a liquid form reconstituted from a lyophilized form.

The preferred formulation according to the invention, can be administered by intravenous (i.v.), subcutaneous (s.c.) or any other parental administration means such as those known in the pharmaceutical art.

The formulation according to the invention also comprises the following specific formulations.

A most preferred liquid formulation according to the invention is:

40 mg/mL huMAb-IGF-1R,

0.03% polysorbate 80,

240 mM trehalose,

10 mM methionine,

20 mM L-histidine,

at pH 5.5;
or

40 mg/mL huMAb-IGF-1R,

0.03% polysorbate 20,

240 mM trehalose,

10 mM methionine,

20 mM Acetate,

at pH 5.5;
or

40 mg/mL huMAb-IGF-1R,

0.03% polysorbate 20,

240 mM trehalose,

20 mM L-histidine,

at pH 5.5;
or

40 mg/mL huMAb-IGF-1R,

0.03% polysorbate 80,

150 mM arginine HCl,

20 mM L-histidine,

at pH 5.5;
or

40 mg/mL huMAb-IGF-1R,

0.03% polysorbate 80,

240 mM trehalose,

20 mM L-histidine,

at pH 5.5;
or

40 mg/mL huMAb-IGF-1R,

20 mM L-histidine,

at pH 5.5;
or

40 mg/mL huMAb-IGF-1R,

20 mM L-histidine,

at pH 6.5;

about 40 mg/mL huMAb-IGF-1R,

about 240 mM Sucrose, and

about 0.03 of polysorbate 20 or polysorbate 80, and

about 20 mM of L-histidine,

optionally 10 mM methionine.

at a pH of about 5.5.

A most preferred lyophilized formulation according to the invention is:

40 mg/mL huMAb-IGF-1R,

0.03% polysorbate 20,

240 mM trehalose,

20 mM L-histidine buffer,

at pH 5.5;
or

40 mg/mL huMAb-IGF-1R,

0.03% polysorbate 80,

240 mM trehalose,

10 mM methionine,

20 mM L-histidine buffer,

at pH 5.5.

Also preferred is the use of a formulation according to the invention for the preparation of a medicament useful for treating diseases modulated by the IGF-IR receptor. In a preferred use according to the invention, the disease is selected from the group consisting of breast cancer, colorectal cancer, non-small cell lung cancer (NSCLC) and prostate cancer or Ewing sarcoma.

The formulation of the invention can further comprise one or more of the following ingredients: antioxidants, ascorbic acid, glutathione, preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); cyclodextrin, e.g. hydroxypropyl-β-cyclodextrin, sulfobutylethyl-β-cyclodextrin, β-cyclodextrin, polyethyleneglycol, e.g. PEG 3000, 3350, 4000, or 6000; low molecular weight (less than about 10 residues) polypeptides; proteins, such as serum albumin, gelatin, or immunoglobulins; chelating agents such as EDTA; salt-forming counter-ions such as sodium; and metal complexes (e.g. Zn-protein complexes).

The formulation of the invention can further comprise one or more stabilizers as defined hereinabove and ingredients also known in the art as “lyoprotectants” such as sugars, sugar alcohols, amino acids and dextrans as known in the art.

In a most preferred embodiment of the formulation according to this invention, the formulation is a liquid form and comprises:

40 mg/mL huMAb-IGF-1R,

0.03% polysorbate 80,

240 mM trehalose,

10 mM methionine, and

20 mM L-histidine

at pH 5.5

This formulation shows good stability upon storage for approximately 6 months at 2-8° C. and 25° C. without formation of visible particles. Shaking and multiple freezing-thawing steps were applied to the liquid formulation to simulate physical stress conditions that potentially occur during manufacturing or transportation of the drug product.

The invention of a liquid formulation according to this embodiment is advantageous as it will facilitate ease of use for the health care provider as no reconstitution step is required and with the higher protein concentration fewer vials per patient will be required.

### EXAMPLES

Liquid and lyophilized drug product formulations for intravenous administration according to the invention were developed as follows.

### Example 1

### Preparation of Liquid Formulations

huMAb-IGF-1R prepared and obtained as disclosed in WO2005/005635 was provided at a concentration of approximately 55 to 65 mg/mL in a 20 mM histidine buffer at a pH of approximately 5.5.

For the preparation of the liquid formulations huMAb-IGF-1R was buffer-exchanged against a diafiltration buffer containing the anticipated buffer composition and where required, concentrated by diafiltration to an antibody concentration of approximately 70 mg/mL. After completion of the diafiltration operation, the excipients (e.g. trehalose) were added as stock solutions to the antibody solution. The surfactant was then added as a 50 to 200-fold stock solution. Finally the protein concentration was adjusted with a buffer to the final huMAb-IGF-1R concentration of approximately 40 mg/mL.

All formulations were sterile-filtered through 0.22 μm low protein binding filters and aseptically filled into sterile 6 mL glass vials closed with ETFE (Copolymer of ethylene and tetrafluoroethylene)-coated rubber stoppers and alucrimp caps. The fill volume was approximately 2.4 mL. These formulations were stored at different climate conditions (5° C., 25° C. and 40° C.) for different intervals of time and stressed by shaking (1 week at a shaking frequency of 200 min⁻¹ at 5° C. and 25° C.) and freeze-thaw stress methods. The samples were analyzed before and after applying the stress tests by the analytical methods 1) UV spectrophotometry, 2) Size Exclusion Chromatography (SEC), 3) by Ion exchange chromatography (IEC), 4) by turbidity of the solution and 5) for visible particles.

UV spectroscopy, used for determination of protein content, was performed on a Perkin Elmer λ35 UV spectrophotometer in a wavelength range from 240 nm to 400 nm. Neat protein samples were diluted to approximately 0.5 mg/mL with the corresponding formulation buffer. The protein concentration was calculated according to equation 1.

$$Protein content = \frac{A ( 280 ) - A ( 320 ) \times dil . factor}{ɛ 〈 {cm}^{2} / mg 〉 \times d 〈 cm 〉} \qquad Equation 1$$

The UV light absorption at 280 nm was corrected for light scattering at 320 nm and multiplied with the dilution factor, which was determined from the weighed masses and densities of the neat sample and the dilution buffer. The numerator was divided by the product of the cuvette's path length d and the extinction coefficient Σ.

Size Exclusion Chromatography (SEC) was used to detect soluble high molecular weight species (aggregates) and low molecular weight hydrolysis products (LMW) in the formulations. The method was performed on a Waters Alliance 2695 HPLC instrument with a Waters W2487 Dual Absorbance Detector and equipped with a TosoHaas TSK-Gel G3000SWXL column. Intact monomer, aggregates and hydrolysis products were separated by an isocratic elution profile, using 0.2M K₂HPO₄/0.25M KCL, pH 7.0 as mobile phase, and were detected at a wavelength of 280 nm.

Ion Exchange Chromatography (IEC) was performed to detect chemical degradation products altering the net charge of huMAb-IGF-1R in the formulations. The method used a suitable HPLC instrument equipped with a UV detector (detection wavelength 220 nm) and a SynChropak WCX column (4.6 mm×250 mm). 10 mM sodium phosphate buffer pH 7.0 in H₂O and 10 mM sodium phosphate buffer pH 7.0+750 mM NaCl were used as mobile phases A and B, respectively, with a flow rate of 1.0 mL/min.

For the determination of the turbidity, opalescence was measured in FTU (turbidity units) using a HACH 2100AN turbidimeter at room temperature.

Samples were analyzed for visible particles by using a Seidenader V90-T visual inspection instrument.
Compositions and Stability Data of Liquid huMAb-IGF-1R Drug Product Formulations according to this Invention

Formulation A is a liquid formulation with the composition 40 mg/mL huMAb-IGF-1R, 20 mM L-histidine, 240 mM trehalose, 10 mM methionine, 0.03% polysorbate 80, at pH 5.5.

| | | Protein | Size Exclusion-HPLC | | | Ion Exchange-HPLC | | | | |
|---|---|---|---|---|---|---|---|---|---|---|
| Storage | Storage | concentration | HMW | Monomer | LMW | Mean Peak | Acidic Peak | Basic Peak | Turbidity | |
| condition | Time | (mg/mL) | (%) | (%) | (%) | (%) | (%) | (%) | (FTU) | Visible particles |
| — | Initial | 40.2 | 0.5 | 99.5 | 0 | 65 | 4 | 31 | 2.9 | Free from particles |
| Shaking | 1 week | 40.7 | 0.6 | 99.4 | 0 | 66 | 4 | 30 | 3.6 | Free from particles |
| 5° C. | | | | | | | | | | |
| Shaking | 1 week | 40.5 | 0.5 | 99.5 | 0 | 71 | 4 | 25 | 3.7 | Free from particles |
| 25° C. | | | | | | | | | | |
| Freezing/ | — | 40.3 | 0.6 | 99.5 | 0 | 66 | 4 | 30 | 3.9 | Essentially free from |
| Thawing | | | | | | | | | | particles |
| (5 cycles) | | | | | | | | | | |
| 5° C. | 8 weeks | 40.4 | 0.6 | 99.4 | 0 | 67 | 4 | 29 | 3.7 | Free from particles |
| | 13 weeks | 40.5 | 0.5 | 99.5 | 0 | 64 | 4 | 32 | 3.6 | Free from particles |
| | 26 weeks | 40.9 | 0.6 | 99.3 | 0.1 | 71 | 3 | 26 | 3.8 | Free from particles |
| 25° C. | 8 weeks | 40.5 | 0.6 | 99.3 | 0.1 | 89 | 5 | 7 | 3.6 | Free from particles |
| | 13 weeks | 40.3 | 0.6 | 99.3 | 0.2 | 65 | 4 | 31 | 3.7 | Free from particles |
| | 26 weeks | 40.4 | 0.8 | 98.7 | 0.5 | 89 | 6 | 6 | 3.8 | Free from particles |
| 40° C. | 8 weeks | 40.6 | 0.7 | 98.2 | 1.1 | 85 | 7 | 6 | 4.3 | Free from particles |
| | 13 weeks | 39.8 | 0.8 | 97.6 | 1.7 | 59 | 11 | 30 | 3.9 | Free from particles |

Formulation B is a liquid formulation with the composition 40 mg/mL huMAb-IGF-1R, 20 mM acetate, 240 mM trehalose, 0.03% polysorbate 20, at pH 5.5.

| | | Protein | Size Exclusion-HPLC | | | Ion Exchange-HPLC | | | | |
|---|---|---|---|---|---|---|---|---|---|---|
| Storage | Storage | concentration | HMW | Monomer | LMW | Mean Peak | Acidic Peak | Basic Peak | Turbidity | |
| condition | Time | (mg/mL) | (%) | (%) | (%) | (%) | (%) | (%) | (FTU) | Visible particles |
| — | Initial | 39.5 | 0.5 | 99.5 | 0 | 65 | 4 | 31 | 2.9 | Free from particles |
| Shaking | 1 week | 40.4 | 0.6 | 99.4 | 0 | 68 | 4 | 29 | 4.2 | Free from particles |
| 5° C. | | | | | | | | | | |
| Shaking | 1 week | 40.1 | 0.6 | 99.4 | 0 | 72 | 4 | 24 | 3.8 | With a few particles |
| 25° C. | | | | | | | | | | |
| Freezing/ | — | 39.9 | 0.6 | 99.4 | 0 | 66 | 4 | 30 | 3.5 | Essentially free from |
| Thawing | | | | | | | | | | particles |
| (5 cycles) | | | | | | | | | | |
| 5° C. | 8 weeks | 40.3 | 0.6 | 99.4 | 0 | 67 | 4 | 29 | 3.4 | Free from particles |
| | 13 weeks | 39.7 | 0.6 | 99.5 | 0 | 68 | 4 | 28 | 3.6 | Free from particles |
| | 26 weeks | 39.5 | 0.7 | 99.2 | 0.1 | 72 | 4 | 25 | 3.9 | Essentially free from |
| | | | | | | | | | | particles |
| 25° C. | 8 weeks | 39.9 | 0.7 | 99.2 | 0.1 | 89 | 4 | 7 | 3.8 | Free from particles |
| | 13 weeks | 39.5 | 0.7 | 99.2 | 0.2 | 88 | 5 | 7 | 3.5 | Free from particles |
| | 26 weeks | 39.7 | 0.9 | 98.5 | 0.5 | 89 | 5 | 6 | 3.7 | Free from particles |
| 40° C. | 8 weeks | 40.6 | 1.1 | 98.1 | 0.9 | 86 | 6 | 6 | 3.7 | Free from particles |
| | 13 weeks | 39.2 | 1.3 | 97.2 | 1.5 | 76 | 18 | 6 | 3.7 | Free from particles |

Formulation C is a liquid formulation with the composition 40 mg/mL huMAb-IGF-1R, 20 mM L-histidine, 240 mM trehalose, 0.03% polysorbate 20, at pH 5.5.

| | | Protein | Size Exclusion-HPLC | | | Ion Exchange-HPLC | | | | |
|---|---|---|---|---|---|---|---|---|---|---|
| Storage | Storage | concentration | HMW | Monomer | LMW | Mean Peak | Acidic Peak | Basic Peak | Turbidity | |
| condition | Time | (mg/mL) | (%) | (%) | (%) | (%) | (%) | (%) | (FTU) | Visible particles |
| — | Initial | 40.0 | 0.5 | 99.5 | 0 | 65 | 4 | 31 | 2.8 | Free from particles |
| Shaking | 1 week | 40.4 | 0.7 | 99.4 | 0 | 66 | 4 | 30 | 3.5 | Free from particles |
| 5° C. | | | | | | | | | | |
| Shaking | 1 week | 39.9 | 0.5 | 99.5 | 0 | 71 | 4 | 25 | 3.9 | Essentially free from |
| 25° C. | | | | | | | | | | particles |
| Freezing/ | — | 40.1 | 0.6 | 99.4 | 0 | 66 | 4 | 30 | 4.2 | Essentially free from |
| Thawing | | | | | | | | | | particles |
| (5 cycles) | | | | | | | | | | |
| 5° C. | 8 weeks | 40.2 | 0.6 | 99.4 | 0 | 67 | 4 | 29 | 3.8 | Free from particles |
| | 13 weeks | 40.2 | 0.5 | 99.5 | 0 | 67 | 4 | 29 | 3.4 | Free from particles |
| | 26 weeks | 40.1 | 0.8 | 99.1 | 0.1 | 71 | 3 | 25 | 3.6 | Free from particles |
| 25° C. | 8 weeks | 40.5 | 0.7 | 99.2 | 0.1 | 89 | 5 | 7 | 4.3 | Free from particles |
| | 13 weeks | 40.0 | 0.6 | 99.2 | 0.2 | 88 | 5 | 7 | 3.5 | Free from particles |
| | 26 weeks | 41.3 | 0.8 | 98.6 | 0.5 | 88 | 6 | 6 | 3.7 | Essentially free from |
| | | | | | | | | | | particles |
| 40° C. | 8 weeks | 39.9 | 1.0 | 98.0 | 1.1 | 85 | 7 | 6 | 4.6 | Free from particles |
| | 13 weeks | 40.0 | 1.1 | 97.2 | 1.7 | 74 | 21 | 6 | 4.0 | Free from particles |

Formulation D is a liquid formulation with the composition 40 mg/mL huMAb-IGF-1R, 20 mM L-histidine, 150 mM arginine-HCl, 0.03% polysorbate 80, at pH 5.5.

| | | Protein | Size Exclusion-HPLC | | | Ion Exchange-HPLC | | | | |
|---|---|---|---|---|---|---|---|---|---|---|
| Storage | Storage | concentration | HMW | Monomer | LMW | Mean Peak | Acidic Peak | Basic Peak | Turbidity | |
| condition | Time | (mg/mL) | (%) | (%) | (%) | (%) | (%) | (%) | (FTU) | Visible particles |
| — | Initial | 39.9 | 0.5 | 99.5 | 0 | 65 | 4 | 32 | 10.1 | Essentially free from |
| | | | | | | | | | | particles |
| Shaking | 1 week | 40.4 | 0.6 | 99.3 | 0.1 | 66 | 4 | 30 | 11.0 | Free from particles |
| 5° C. | | | | | | | | | | |
| Shaking | 1 week | 40.4 | 0.6 | 99.4 | 0 | 72 | 4 | 25 | 18.3 | With many particles |
| 25° C. | | | | | | | | | | |
| Freezing/ | — | 40.5 | 0.6 | 99.4 | 0 | 66 | 4 | 30 | 11.4 | Free from particles |
| Thawing | | | | | | | | | | |
| (5 cycles) | | | | | | | | | | |
| 5° C. | 8 weeks | 40.1 | 0.7 | 99.3 | 0 | 67 | 4 | 29 | 11.4 | Free from particles |
| | 13 weeks | 40.1 | 0.6 | 99.4 | 0 | 68 | 4 | 29 | 11.6 | Free from particles |
| | 26 weeks | 41.7 | 0.9 | 99.1 | 0.1 | 72 | 3 | 25 | 11.8 | Free from particles |
| 25° C. | 8 weeks | 41.1 | 0.7 | 99.1 | 0.1 | 89 | 4 | 7 | 15 | Free from particles |
| | 13 weeks | 40.5 | 0.8 | 99.0 | 0.2 | 88 | 5 | 7 | 16.9 | With many particles |
| | 26 weeks | 40.6 | 1.1 | 98.3 | 0.6 | 89 | 6 | 6 | 16.6 | Free from particles |
| 40° C. | 8 weeks | 41.0 | 1.4 | 97.3 | 1.3 | 85 | 8 | 5 | 21 | With many particles |
| | 13 weeks | 41.3 | 0.7 | 98.0 | 1.3 | 59 | 37 | 5 | 23.3 | With many particles |

Formulation E is a liquid formulation with the composition 40 mg/mL huMAb-IGF-1R, 20 mM L-histidine, 240 mM trehalose, 0.03% polysorbate 80, at pH 5.5.

| | | Protein | Size Exclusion-HPLC | | | Ion Exchange-HPLC | | | | |
|---|---|---|---|---|---|---|---|---|---|---|
| Storage | Storage | concentration | HMW | Monomer | LMW | Mean Peak | Acidic Peak | Basic Peak | Turbidity | |
| condition | Time | (mg/mL) | (%) | (%) | (%) | (%) | (%) | (%) | (FTU) | Visible particles |
| — | Initial | 40.2 | 0.5 | 99.5 | 0 | 65 | 4 | 31 | 3.0 | Free from particles |
| Shaking | 1 week | 40.3 | 0.6 | 99.4 | 0 | 66 | 4 | 30 | 4.2 | Free from particles |
| 5° C. | | | | | | | | | | |
| Shaking | 1 week | 39.5 | 0.6 | 99.4 | 0 | 72 | 4 | 25 | 4.0 | Free from particles |
| 25° C. | | | | | | | | | | |
| Freezing/ | — | 40.3 | 0.6 | 99.4 | 0 | 66 | 4 | 30 | 3.8 | Free from particles |
| Thawing | | | | | | | | | | |
| (5 cycles) | | | | | | | | | | |
| 5° C. | 8 weeks | 40.8 | 0.6 | 99.4 | 0 | 67 | 4 | 29 | 3.8 | Free from particles |
| | 13 weeks | 40.4 | 0.5 | 99.5 | 0 | 67 | 4 | 29 | 3.6 | Free from particles |
| | 26 weeks | 40.2 | 0.7 | 99.2 | 0.1 | 71 | 4 | 25 | 3.46 | Free from particles |
| 25° C. | 8 weeks | 38.5 | 0.7 | 99.2 | 0.1 | 89 | 5 | 7 | 3.1 | Free from particles |
| | 13 weeks | 40.7 | 0.7 | 99.2 | 0.2 | 88 | 5 | 7 | 3.6 | Free from particles |
| | 26 weeks | 40.8 | 1.0 | 98.4 | 0.6 | 87 | 7 | 6 | 3.9 | Free from particles |
| 40° C. | 8 weeks | 40.2 | 1.4 | 97.5 | 1.1 | 83 | 11 | 5 | 4.0 | Free from particles |
| | 13 weeks | 40.1 | 1.5 | 96.8 | 1.8 | 72 | 22 | 6 | 4.2 | Free from particles |

Further experiments using the Umetrics DoE software (Modde) were performed including following variations:

variations in pH from about 5.0 to 6.0

variations in protein content from about 34 mg/mL to approximately 46 mg/mL

variations in surfactant from about 0.02% to 0.04%

variations in stabilizer (trehalose) from about 215 to 265 mM

The preparation of the samples occurred as described above. These formulations were stored at different climate conditions (5° C., 25° C. and 40) for different intervals of time. The samples were analyzed by the analytical methods 1) UV spectrophotometry, 2) Size Exclusion Chromatography (SEC), 3) by Ion exchange chromatography (IEC), 4) by turbidity of the solution and 5) for visible particles.

The 3 month storage data demonstrate that pH has an impact on soluble aggregates (Size Exclusion-HPLC, HMWs) and fragments (Size Exclusion-HPLC, LMWs). The results show that even at 40° C. the formulation is sufficiently stable even when pH is varied from about pH 5.0 to about pH 6.0, protein concentration is varied from about 34 to about 46 mg/mL, surfactant concentration is varied from about 0.02 to about 0.04% (w/v) and stabilizer concentration is varied from about 215 to about 265 mM.

### Example 2

### Preparation of Lyophilized Formulation

Solutions of approximately 40 mg/ml huMAb-IGF-1R were prepared as described above for liquid formulations. All formulations were sterile filtered through 0.22 μm filters and aseptically aliquoted into sterile 20 mL glass vials. The vials were partly closed with ETFE (Copolymer of ethylene and tetrafluoroethylene)-coated rubber stoppers suitable for the use in lyophilization processes and lyophilized using the freeze-drying cycle reported in Table 1.

TABLE 1
Freeze-drying Cycle

| | | Ramp | | Vacuum |
|---|---|---|---|---|
| | Shelf temperature | Rate | Hold time | Set point |
| Step | (° C.) | (° C./min) | (min) | (μbar) |
| Pre-cooling | 5° C. | 0.0 | 60 | — |
| Freezing | −40° C. | 1.0 | 120 | — |
| Primary Drying | −25° C. | 0.5 | 4560 | 80 |
| Secondary Drying | +25° C. | 0.2 | 300 | 80 |

The product was first cooled from room temperature to approx 5° C. (pre-cooling), followed by a freezing step at −40° C. with a plate cooling rate of approximately 1° C./min, followed by a holding step at −40° C. for about 2 hours . The first drying step was performed at a plate temperature of approximately −25° C. and a chamber pressure of approximately 80 μbar for about 76 hours. Subsequently, the second drying step started with a temperature ramp of 0.2° C./min from −25° C. to 25° C., followed by a holding step at 25° C. for at least 5 hours at a chamber pressure of approximately 80 μbar.

Lyophilization was carried out in a Usifroid SMH-90 LN2 freeze-dryer (Usifroid, Maurepas, France) or a LyoStar II Freeze-dryer (FTS Systems, Stone Ridge, N.Y., USA). The freeze-dried samples were stored at different climate conditions (5° C., 25° C. and 40° C.) for different intervals of time. The lyophilized vials were reconstituted to a final volume of 5.3 mL with water for injection (WFI) yielding an isotonic formulation with an antibody concentration of approximately 40 mg/mL. The reconstitution time of the freeze-dried cakes was around 1 min. Analysis of the reconstituted samples was performed after a 24 hour incubation period of the reconstituted liquid sample at 25° C.

The samples were analyzed by the analytical methods 1) UV spectrophotometry, 2) Size Exclusion Chromatography (SEC), 3) by Ion exchange chromatography (IEC), 4) by turbidity of the solution and 3) for visible particles.

UV spectroscopy, used for determination of protein content, was performed on a Perkin Elmer λ UV spectrophotometer in a wavelength range from 240 nm to 400 nm. Neat protein samples were diluted to approximately 0.5 mg/mL with the corresponding formulation buffer. The protein concentration was calculated according to equation 1.

$$Protein content = \frac{A ( 280 ) - A ( 320 ) \times dil . factor}{ɛ 〈 {cm}^{2} / mg 〉 \times d 〈 cm 〉} \qquad Equation 1$$

The UV light absorption at 280 nm was corrected for light scattering at 320 nm and multiplied with the dilution factor, which was determined from the weighed masses and densities of the neat sample and the dilution buffer. The numerator was divided by the product of the cuvette's path length d and the extinction coefficient Σ.

Size Exclusion Chromatography (SEC) was used to detect soluble high molecular weight species (aggregates) and low molecular weight hydrolysis products (LMW) in the formulations. The method was performed on a Waters Alliance 2695 HPLC instrument with a Waters W2487 Dual Absorbance Detector and equipped with a TosoHaas TSK-Gel G3000SWXL column. Intact monomer, aggregates and hydrolysis products were separated by an isocratic elution profile, using 0.2 M K₂HPO₄/0.25 M KCL, pH 7.0 as mobile phase, and were detected at a wavelength of 280 nm.

Ion Exchange Chromatography (IEC) was performed to detect chemical degradation products altering the net charge of HuMAb-IGF-1R in the formulations. The method used a suitable HPLC instrument equipped with a UV detector (detection wavelength 220 nm) and a SynChropak WCX column (4.6 mm×250 mm). 10 mM sodium phosphate buffer pH 7.0 in H₂O and 10 mM sodium phosphate buffer pH 7.0+750 mM NaCl were used as mobile phases A and B, respectively, with a flow rate of 1.0 mL/min.

For the determination of the turbidity, opalescence was measured in FTU (turbidity units) using a HACH 2100AN turbidimeter at room temperature.

Compositions and stability data of liquid HuMAb-IGF-1R drug product formulations according to this invention Formulation F is a lyophilized formulation with the composition 40 mg/mL huMAb-IGF-1R, 20 mM L-histidine, 240 mM trehalose, 0.03% polysorbate 20, at pH 5.5.

| | | Protein | Size Exclusion-HPLC | | | Ion Exchange-HPLC | | | |
|---|---|---|---|---|---|---|---|---|---|
| Storage | Storage | concentration | HMW | Monomer | LMW | Mean Peak | Acidic Peak | Basic Peak | Turbidity |
| condition | Time | (mg/mL) | (%) | (%) | (%) | (%) | (%) | (%) | (FTU) |
| — | Initial | 40.5 | 0.4 | 99.6 | 0 | 65 | 4 | 31 | 3.7 |
| 5° C. | 8 weeks | 41.6 | 0.6 | 99.4 | 0 | 65 | 4 | 31 | 3.7 |
| | 13 weeks | 40.6 | 0.5 | 99.5 | 0 | 65 | 4 | 31 | 3.6 |
| | 26 weeks | 41.2 | 0.6 | 99.3 | 0.1 | 66 | 3 | 31 | 3.8 |
| 25° C. | 8 weeks | 40.7 | 0.7 | 99.3 | 0 | 61 | 4 | 9 | 3.6 |
| | 13 weeks | 40.6 | 0.6 | 99.4 | 0 | 59 | 4 | 30 | 3.4 |
| | 26 weeks | 41.1 | 0.8 | 99.2 | 0 | 59 | 3 | 30 | 3.7 |
| 40° C. | 8 weeks | 40.8 | 0.9 | 99.1 | 0 | 89 | 3 | 8 | 3.8 |
| | 13 weeks | 40.0 | 0.9 | 99.1 | 0 | 57 | 15 | 29 | 3.7 |

Formulation G is a lyophilized formulation with the composition 40 mg/mL huMAb-IGF-1R, 20 mM L-histidine, 240 mM trehalose, 10 mM methionine, 0.03% polysorbate 80, at pH 5.5.

### Example 3

### Preparation of Liquid Formulations for pH Study

huMAb-IGF-1R prepared and obtained as disclosed in WO2005/005635 was provided at a concentration of approximately 70 mg/mL in a 20 mM histidine buffer at a pH of approximately 5.5.

For the preparation of the liquid formulations for the pH study huMAb-IGF-1R was buffer-exchanged against a diafiltration buffer containing the anticipated buffer composition and the protein concentration was adjusted with a buffer to the final huMAb-IGF-1R concentration of approximately 40 mg/mL.

All formulations were sterile-filtered through 0.22 μm low protein binding filters and aseptically filled into sterile 6 mL glass vials closed with ETFE (Copolymer of ethylene and tetrafluoroethylene)-coated rubber stoppers and alucrimp caps. The fill volume was approximately 2.4 mL. These formulations were stored at different climate conditions (5° C., 25° C. and 40° C.) for different intervals of time and stressed by shaking (1 week at a shaking frequency of 200 min⁻¹ at 5° C. and 25° C.) and freeze-thaw stress methods. The samples were analyzed before and after applying the stress tests by the analytical methods 1) UV spectrophotometry, 2) Size Exclusion Chromatography (SEC), 3) by Ion exchange chromatography (IEC), 4) by turbidity of the solution and 5) for visible particles.

UV spectroscopy, used for determination of protein content, was performed on a Perkin Elmer λ35 UV spectrophotometer in a wavelength range from 240 nm to 400 nm. Neat protein samples were diluted to approximately 0.5 mg/mL with the corresponding formulation buffer. The protein concentration was calculated according to equation 1.

$$Protein content = \frac{A ( 280 ) - A ( 320 ) \times dil . factor}{ɛ 〈 {cm}^{2} / mg 〉 \times d 〈 cm 〉} \qquad Equation 1$$

The UV light absorption at 280 nm was corrected for light scattering at 320 nm and multiplied with the dilution factor, which was determined from the weighed masses and densities of the neat sample and the dilution buffer. The numerator was divided by the product of the cuvette's path length d and the extinction coefficient Σ.

Size Exclusion Chromatography (SEC) was used to detect soluble high molecular weight species (aggregates) and low molecular weight hydrolysis products (LMW) in the formulations. The method was performed on a Waters Alliance 2695 HPLC instrument with a Waters W2487 Dual Absorbance Detector and equipped with a TosoHaas TSK-Gel G3000SWXL column. Intact monomer, aggregates and hydrolysis products were separated by an isocratic elution profile, using 0.2 M K₂HPO₄/0.25 M KCL, pH 7.0 as mobile phase, and were detected at a wavelength of 280 nm.

Ion Exchange Chromatography (IEC) was performed to detect chemical degradation products altering the net charge of HuMAb-IGF-1R in the formulations. The method used a suitable HPLC instrument equipped with a UV detector (detection wavelength 220 nm) and a SynChropak WCX column (4.6 mm×250 mm). 10 mM sodium phosphate buffer pH 7.0 in H₂O and 10 mM sodium phosphate buffer pH 7.0+750 mM NaCl were used as mobile phases A and B, respectively, with a flow rate of 1.0 mL/min.

For the determination of the turbidity, opalescence was measured in FTU (turbidity units) using a HACH 2100AN turbidimeter at room temperature.

Samples were analyzed for visible particles by using a Seidenader V90-T visual inspection instrument.

Formulation H is a liquid formulation with the composition 40 mg/mL huMAb-IGF-1R, 20 mM L-histidine at pH 5.5.

| | | Protein | Size Exclusion-HPLC | | | Ion Exchange-HPLC | | | | |
|---|---|---|---|---|---|---|---|---|---|---|
| Storage | Storage | concentration | HMW | Monomer | LMW | Mean Peak | Acidic Peak | Basic Peak | Turbidity | |
| condition | Time | (mg/mL) | (%) | (%) | (%) | (%) | (%) | (%) | (FTU) | Visible particles |
| — | Initial | 39.0 | 0.4 | 99.6 | 0 | 69 | 4 | 27 | 3.9 | Essentially free from |
| | | | | | | | | | | particles |
| Shaking | 1 week | 39.9 | 0.4 | 99.6 | 0 | 67 | 4 | 29 | 4.6 | With many particles |
| 5° C. | | | | | | | | | | |
| Shaking | 1 week | 39.7 | 0.5 | 99.6 | 0 | 73 | 4 | 23 | 6.6 | With many particles |
| 25° C. | | | | | | | | | | |
| Freezing/ | — | 39.7 | 0.9 | 99.1 | 0 | 67 | 4 | 29 | 6.1 | With many particles |
| Thawing | | | | | | | | | | |
| (5 cycles) | | | | | | | | | | |
| 5° C. | 8 weeks | 39.5 | 0.3 | 99.7 | 0 | 69 | 4 | 27 | 3.7 | Essentially free from |
| | | | | | | | | | | particles |
| | 13 weeks | 39.2 | 0.6 | 99.4 | 0 | 72 | 4 | 24 | 5.5 | Essentially free from |
| | | | | | | | | | | particles |
| 25° C. | 8 weeks | 40.1 | 0.5 | 99.4 | 0.1 | 88 | 5 | 7 | 5.0 | With many particles |
| | 13 weeks | 39.1 | 0.9 | 98.9 | 0.3 | 87 | 6 | 7 | 5.9 | Essentially free from |
| | | | | | | | | | | particles |
| 40° C. | 8 weeks | 39.5 | 0.9 | 99.0 | 5.0 | 85 | 10 | 6 | 4.1 | With many particles |
| | 13 weeks | 38.8 | 1.8 | 88.5 | 9.7 | 58 | 39 | 4 | 5.2 | With many particles |

Formulation I is a liquid formulation with the composition 40 mg/mL huMAb-IGF-1R and 20 mM L-histidine at pH 6.5.

| | | Protein | Size Exclusion-HPLC | | | Ion Exchange-HPLC | | | | |
|---|---|---|---|---|---|---|---|---|---|---|
| Storage | Storage | concentration | HMW | Monomer | LMW | Main Peak | Acidic Peak | Basic Peak | Turbidity | |
| condition | Time | (mg/mL) | (%) | (%) | (%) | (%) | (%) | (%) | (FTU) | Visible particles |
| — | Initial | 40.2 | 0.8 | 99.2 | 0 | 65 | 4 | 23 | 3.3 | practically free |
| Shaking | 1 week | 40.4 | 1.1 | 98.9 | 0 | 82 | 4 | 13 | 3.2 | with many particles |
| 5° C. | | | | | | | | | | |
| Shaking | 1 week | 39.3 | 1.1 | 98.9 | 0 | 76 | 6 | 18 | 12.3 | with many particles |
| 25° C. | | | | | | | | | | |
| Freezing/ | — | 38.0 | 1.2 | 99.9 | 0 | 78 | 6 | 17 | 5.8 | with many particles |
| Thawing | | | | | | | | | | |
| (5 cycles) | | | | | | | | | | |
| 5° C. | 8 weeks | 40.3 | 1.0 | 99.0 | 0 | 71 | 5 | 24 | 2.5 | practically free |
| | 19 weeks | 41.0 | 1.4 | 98.6 | 0 | 72 | 7 | 22 | 2.6 | with many particles |
| 25° C. | 8 weeks | 40.6 | 1.8 | 98.1 | 0.1 | 94 | 3 | 4 | 3.4 | with many particles |
| | 19 weeks | 39.4 | 2.4 | 97.3 | 0.3 | 85 | 11 | 4 | 3.0 | with a few particles |
| 40° C. | 8 weeks | 40.4 | 2.5 | 94.7 | 2.9 | 84 | 13 | 3 | 3.3 | with many particles |
| | 19 weeks | 39.9 | 4.8 | 88 | 7 | 52 | 45 | 3 | 4.2 | with many particles |

Formulation J is a liquid formulation with the composition 40 mg/mL huMab IGF-1R, 20 mM L-histidine, 240 mM sucrose, 10 mM Methionine, 0.03% polysorbate 80, at pH 5.5.

| | | Protein | Size Exclusion-HPLC | | | Ion Exchange-HPLC | | | | |
|---|---|---|---|---|---|---|---|---|---|---|
| Storage | Storage | concentration | HMW | Monomer | LMW | Mean | Acidic | Basic | Turbidity | |
| condition | Time | (mg/mL) | (%) | (%) | (%) | Peak (%) | Peak (%) | Peak (%) | (FTU) | Visible particles |
| — | Initial | 40.5 | 0.9 | 99.1 | 0.1 | 62.9 | 5.9 | 31.3 | 4.1 | Free from particles |
| Shaking | 1 week | 40.9 | 0.9 | 99.1 | 0 | 66.9 | 6.0 | 27.1 | 3.8 | Essentially free |
| 5° C. | | | | | | | | | | from particles |
| Shaking | 1 week | 40.6 | 0.9 | 99.1 | 0 | 68.6 | 6.1 | 25.8 | 3.9 | Free from particles |
| 25° C. | | | | | | | | | | |
| Freezing/ | — | 40.9 | 0.9 | 99.1 | 0 | 66.1 | 6.1 | 27.8 | 3.6 | Free from particles |
| Thawing | | | | | | | | | | |
| (5 cycles) | | | | | | | | | | |
| 25° C. | 4 weeks | 41.0 | 0.8 | 99.0 | 0.2 | 76.1 | 6.0 | 17.8 | 4.0 | Essentially free |
| | | | | | | | | | | from particles |
| 40° C. | 4 weeks | 40.8 | 0.8 | 98.4 | 0.9 | 84.4 | 8.7 | 6.3 | 4.6 | Free from particles |

What is claimed is:

1. A formulation comprising:
about 20 to about 60 mg/mL huMAb-IGF-1R,
about 5 to about 50 mM of a buffer,
at a pH from about 4.5 to about 7.0.

2. The formulation according to claim 1 comprising:
about 30 to about 50 mg/mL huMAb-IGF-1R,
about 10 to about 30 mM of a buffer,
at a pH from about 5.0 to about 6.5.

3. The formulation according to claim 2 further comprising about 0.01% to about 0.06% of at least one surfactant.

4. The formulation according to any one of claim 3 further comprising at least one stabilizer.

5. The formulation according to claim 4, wherein the stabilizers are selected from the group of sugars and amino acids.

6. The formulation according to claim 5, wherein the stabilizer is trehalose.

7. The formulation according to claim 6, wherein the formulation comprises about 200 mM to about 300 mM trehalose.

8. The formulation according to claim 4, wherein the stabilizer is methionine.

9. The formulation according to claim 8, wherein the formulation comprises about 5 mM to about 30 mM methionine.

10. The formulation according to claim 5, wherein the formulation comprises trehalose and methionine.

11. The formulation according to claim 10, wherein the formulation comprises about 230 mM to 250 mM trehalose and about 5 mM to 15 mM methionine.

12. The formulation according to claim 1 comprising:
about 35 to about 45 mg/mL huMAb-IGF-1R,
at least one further stabilizer, and
about 0.02 to about 0.04% of at least one surfactant, and
about 15 to about 25 mM of a buffer,
at a pH from about 5.0 to about 6.0.

13. The formulation according to claim 12, wherein the formulation comprises about 230 mM to 250 mM trehalose and about 5 mM to 15 mM methionine.

14. The formulation according to claim 1 comprising:
about 40 mg/mL huMAb-IGF-1R,
about 240 mM trehalose, and
about 0.03 of polysorbate 20 or polysorbate 80, and
about 20 mM of a L-histidine,
at a pH of about 5.5.

15. The formulation according to claim 14 further comprising about 10 mM methionine.

16. The formulation according to claim 1, which is in a liquid form, in a lyophilized form or in a liquid form reconstituted from a lyophilized form.

17. The liquid formulation according to claim 1, comprising:
40 mg/mL huMAb-IGF-1R,
0.03% polysorbate 80,
240 mM trehalose,
10 mM methionine,
20 mM L-histidine ,
at pH 5.5;
or
40 mg/mL huMAb-IGF-1R,
0.03% polysorbate 20,
240 mM trehalose,
10 mM methionine,
20 mM acetate,
at pH 5.5;
or
40 mg/mL huMAb-IGF-1R,
0.03% polysorbate 20,
240 mM trehalose,
20 mM L-histidine,
at pH 5.5;
or
40 mg/mL huMAb-IGF-1R,
0.03% polysorbate 80,
150 mM arginine HCl,
20 mM L-histidine,
at pH 5.5;
or
40 mg/mL huMAb-IGF-1R,
0.03% polysorbate 80,
240 mM trehalose,
20 mM L-histidine,
at pH 5.5;
or
about 40 mg/mL huMAb-IGF-1R,
about 240 mM Sucrose, and
about 0.03 of polysorbate 20 or polysorbate 80, and
about 20 mM of L-histidine,
at a pH of about 5.5;
or
about 40 mg/mL huMAb-IGF-1R,
about 240 mM Sucrose, and
about 0.03 of polysorbate 20 or polysorbate 80, and
about 20 mM of L-histidine,
about 10 mM methionine,
at a pH of about 5.5;
or
40 mg/mL huMAb-IGF-1R,
20 mM L-histidine,
at pH 5.5;
or
40 mg/mL huMAb-IGF-1R,
20 mM L-histidine,
at pH 6.5.

19. The lyophilized formulation according to any one of claims 1 to 17, wherein it is:
40 mg/mL huMAb-IGF-1R,
0.03% polysorbate 20,
240 mM trehalose,
20 mM L-histidine,
at pH 5.5;
or
40 mg/mL huMAb-IGF-1R,
0.03% polysorbate 80,
240 mM trehalose,
10 mM methionine,
20 mM L-histidine,
at pH 5.5.
